(19) United States
(12) Patent Application Publication
(10) Pub. No.: US 2025/0232861 A1
(43) Pub. Date: Jul. 17, 2025

(54) METHOD OF PAIRING THERAPY DEVICES USING SHARED SECRETS, AND RELATED SYSTEMS, METHODS AND DEVICES

(71) Applicant: Bigfoot Biomedical, Inc., Milpitas, CA (US)

(72) Inventors: Aaron KIM, Milpitas, CA (US); Bryan MAZLISH, Palo Alto, CA (US)

(21) Appl. No.: 18/989,344

(22) Filed: Dec. 20, 2024

Related U.S. Application Data
(63) Continuation of application No. 16/667,876, filed on Oct. 29, 2019, now Pat. No. 12,205,699.
(60) Provisional application No. 62/752,493, filed on Oct. 30, 2018.

(51) Int. Cl. G16H 20/17; A61B 5/00; A61B 5/145; A61M 5/142; G16H 10/60; G16H 40/67; H04L 9/08; H04L 9/32

(52) U.S. Cl. CPC G16H 20/17; A61B 5/14532; A61B 5/4839; A61M 5/14244; G16H 10/60; G16H 40/67; H04L 9/0866; H04L 9/3236; A61M 2230/201; H04L 2209/12

(57) ABSTRACT

Systems, methods and devices are described for establishing trusted connections among two or more therapy devices that form, or form part of, a medication therapy system. A, medication delivery electronics may include a first communication interface, a connection manager, and a therapy management application. A first communication interface may be configured to establish and communicate over one or more communication links. A connection manager may be configured to generate a candidate shared secret key and provide the shared key to a first therapy device over a first communication link established by a first communication interface. A candidate shared key may be generated responsive to one or more shared secret parameters. A therapy management application may be configured to receive, using the first communication interface, the first therapy related information from the first therapy device over a first trusted communication link established by the first communication interface responsive to acceptance of a candidate shared secret.

### CROSS-REFERENCE TO RELATED APPLICATION

This application is a continuation of U.S. application Ser. No. 16/667,876, filed Oct. 29, 2019, which claims the benefit under 3S U.S.C. § 119 (e) of U.S. Provisional Patent Application Ser. No. 62/752,493, filed Oct. 30, 2018, the disclosure of which is hereby incorporated herein in its entirety by this reference.

### TECHNICAL FIELD

Embodiments of this disclosure relate, generally, to medication therapy systems, and more specifically, some embodiments relate to systems and methods for establishing trusted connections among two or more therapy devices that form a medication therapy system.

### BACKGROUND

Medication delivery devices are commonly used to deliver medication to the human body, including as part of a medical treatment provided as a therapy for a medical condition. Medication delivery devices such as a transdermal liquid dosing device (e.g., an injection pen), inhalers, and syringes provide a convenient, often reusable means of delivering medication to the human body in fluid or aerosolized form. Infusion pumps also provide a convenient means of delivering medication to the human body in controlled amounts of fluid, and are often used when there is a desire to deliver medication in precisely calculated volumes (large and small) at precise rates and/or intervals. By way of example, medication delivery means may deliver medication intravenously, subcutaneously, arterially, and epidurally (i.e., via the epidural space around the spinal cord). Infusion pumps use a variety of techniques to deliver medication that do not require any or only a limited amount of manual manipulation. By way of example, infusion pumps may use positive displacement (e.g., a peristaltic pump, diaphragm pump, etc.), positive pressure (e.g., a drive system that advances a plunger), reciprocating positive pressure (e.g., plunger pumps that draw medication from a cartridge into a delivery chamber), and more.

One advantage of infusion pumps is that their dimensions may be such that they can be worn “on the body” For example, on-body infusion pumps are sometimes used for subcutaneous delivery of medication—the infusion pump delivers medication via a subcutaneous cannula, and an adhesive patch secures the pump, cannula and any other elements of the infusion set at the infusion site. The adhesive helps maintain the cannula in fluidic communication with the tissue and/or vasculature of the patient so that medication may be delivered to the patient's body.

Medication delivery devices are sometimes used in combination with other therapy related devices to deliver therapeutic amounts of medication. For example, when a treatment is based, at least in part, on an amount of an analyte in a patient's blood stream. An analyte sensor may be used to detect and/or measure amounts of the analyte and provide analyte data to a user, a medication delivery device, or other therapy related devices. A therapy application may store and analyze the analyte data, plan therapy activities, provide and receive information from users (e.g., via an interface), provide recommendations to users (e.g., via an interface), and, in some arrangements, provide instructions to a medication delivery device for medication delivery.

Medication therapies can exact a toll on users that, due to dangers associated with administering external biologically effective medication (e.g, hormone, analgesics, antibiotics, nutrients, etc.), must track, plan, and calculate amounts of medication delivered. So, therapy applications can be a tremendous boon, and off-load some of the cognitive and emotional burden associated with a medication therapy.

### BRIEF DESCRIPTION OF DRAWINGS

While this disclosure concludes with claims particularly pointing out and distinctly claiming specific embodiments, various features and advantages of embodiments within the scope of this disclosure may be more readily ascertained from the following description when read in conjunction with the accompanying drawings, in which:

FIG. 1 is a functional block diagram of a therapy system, which includes at least some therapy devices that are configured to establish trusted connections in accordance with one or more embodiments of the disclosure.

FIG. 2 is a flowchart of a process for establishing trusted communication links among therapy devices, in accordance with one or more embodiments of the disclosure.

FIG. 3A is a functional block diagram of medication therapy electronics configured for establishing trusted communication links in accordance with one or more embodiments of the disclosure.

FIG. 3B is a flow chart of operations for determining a candidate shared secret key using a pre-defined component and a variable component, in accordance with one or more embodiments of the disclosure.

FIG. 4 is a flowchart of a process for establishing a trusted communication link with another therapy related device, in accordance with one or more embodiments of the disclosure.

FIG. 5 is a functional block diagram of a remote therapy management system, in accordance with one or more embodiments of the disclosure.

FIG. 6 is a flowchart of a process for enforcing policies for establishing trusted communication links among therapy devices, in accordance with one or more embodiments of the disclosure.

FIG. 7 is a functional block diagram of an analyte monitoring unit configured for establishing trusted communication links, in accordance with one or more embodiments of the disclosure.

FIG. 8 is a flowchart of a process for establishing trusted communication links with a therapy device, in accordance with one or more embodiments of the disclosure.

FIG. 9 shows a functional block diagram of a therapy system that includes at least some therapy devices that are configured to establish trusted connections in accordance with one or more embodiments of the disclosure.

FIG. 10 shows a flowchart of a process for establishing a trusted communication link with another therapy related device, in accordance with one or more embodiments of the disclosure.

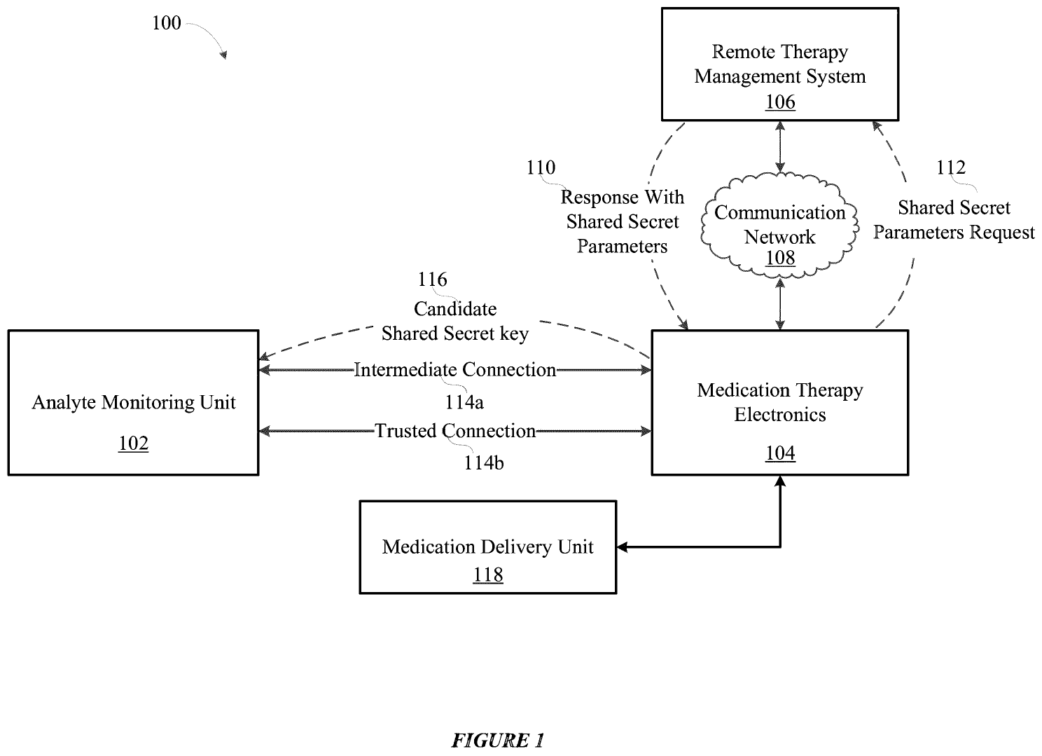

FIG. 1l shows an insulin therapy system configured for pairing therapy devices in accordance with one or more embodiments of the disclosure.

FIG. 12 shows an automated insulin therapy system configured for pairing therapy devices in accordance with one or more embodiments of the disclosure.

FIGS. 13A and 13B show a sequence of operations that are part of a pairing process between a BGM and an insulin delivery device, in accordance with one or more embodiments of the disclosure.

### DETAILED DESCRIPTION

In the following detailed description, reference is made to the accompanying drawings, which form a part hereof, and in which are shown, by way of illustration, specific example embodiments in which the present disclosure may be practiced. These embodiments are described in sufficient detail to enable a person of ordinary skill in the art to practice the present disclosure. However, other embodiments may be utilized, and structural, material, and process changes may be made without departing from the scope of the disclosure.

It should be understood that any reference to an element herein using a designation such as “first.” “second,” and so forth does not limit the quantity or order of those elements, unless such limitation is explicitly stated. Rather, these designations are used herein as a convenient method of distinguishing between two or more elements or instances of an element. Thus, a reference to first and second elements does not mean that only two elements can be employed or that the first element must precede the second element in some manner. Also, unless stated otherwise a set of elements may comprise one or more elements. Likewise, sometimes elements referred to in the singular form may also include one or more instances of the element.

The following description may include examples to help enable one of ordinary skill in the art to practice the disclosed embodiments. The use of the terms “exemplary.” “by example,” and “for example,” means that the related description is explanatory, and though the scope of the disclosure is intended to encompass the examples and legal equivalents, the use of such terms is not intended to limit the scope of an embodiment or this disclosure to the specified components, steps, features, functions, or the like.

Sometimes therapy devices are wirelessly connected to each other to form a therapy system that is configured for medication delivery. For example, an analyte monitoring device may be wirelessly connected to a medication delivery device. While it is common for devices to connect wirelessly to form a system (e.g., a speaker connecting to a media source via BLUETOOTH®), there are specific challenges and concerns that arise in medication delivery, especially ambulatory medication delivery.

For example, for some conditions and therapies, a patient may need to carry an ambulatory medication delivery device (e.g., an infusion pump, injection pen, an inhaler, etc.) with them all times. Even if not necessary, many patients carry their drug delivery devices with them simply for convenience and risk aversion. So, a patient could take an ambulatory medication delivery device loaded with a dangerous medication, literally, anywhere in the world.

Opportunities for mis-delivery and dangerous consequences are higher when patients are being treated by a therapy system that includes a variety of therapy devices communicating with each other. For example, a patient may be treated by a therapy system that includes an analyte monitoring device, a therapy application executing on a mobile device, and a medication delivery device. In a typical operational cycle, the analyte monitoring device sends analyte measurement data to the therapy application. The therapy application performs therapy related analysis, determines changes to a medication therapy regime based on the analyte measurement data, and sends instructions to implement the changes to the medication delivery device. The medication delivery device reconfigures its medication delivery (e.g., dose amount, rate of dosing, etc.) based, at least in part, on the instructions.

If a third-party device (i.e., a therapy device that is not part of the therapy system) connects with any of the therapy devices that form the therapy system, that connection could interfere with the operation of the therapy system. The other device could be part of another therapy system, in which case the connection could interfere with operation of both therapy systems. So, even an inadvertent connection could cause information and instructions to be missed, or erroneous instructions to be sent. Moreover, the third-party device may be a malicious device that intends to capture therapy related information or even hijack operation of the therapy system and interfere with medication delivery (e.g., so called “main-in-the-middle” attacks).

Whether inadvertent or malicious, a misconnection and incorrect operation brings a significant risk of injury and death to users of therapy systems. The risk of an inadvertent or malicious connection are increased if the therapy devices are able to connect to each other and third-party devices using wireless communication links.

As used herein “a connection” and “connected” when used with references to two or more devices means that there is a communication link between two devices. The communication link may be wired, unwired (e.g., using wireless radio-frequency (RF) signals), and combinations thereof. “Connecting” means one or more operations are being taken to establish a communication link or a type of communication link, for example, operations to establish an intermediate communication link, which in turn may be used for performing one or more operations to establish a trusted communication link.

One way to restrict wireless connections is to configure devices to limit at least certain communication to a trusted connection. One of ordinary skill in the art would appreciate a number of ways to establish a trusted connection (when two devices form or have a trusted connection, that is also referred to herein as “pairing” and being “paired”), and one non-limiting example is: discovery, authentication, agreement on a communication protocol, sharing identifiers, agreement on an encryption scheme, and then agreement that the trusted connection is active.

Due to the risk of inadvertent connections and malicious connections, passwords and/or passkeys are often used when establishing trusted connections. For example, a user reads an N-digit code at a first device that is in a pairing mode, and enters the N-digit code at a second device that is in a pairing mode. The second device provides the N-digit code to the first device over a wired or wireless connection. If the first device determines that the displayed N-digit code matches the received N-digit code, then the first device approves the pairing and the first and second devices establish a trusted connection. The N-digit code is an example of a “shared secret,” which is a piece of data, known only to parties involved in setting up the trusted connection. By way of example, a shared secret may be a password, a passphrase, a big number, an array of randomly chosen bytes, and the like. In some cases, both devices will store the shared secret and, periodically, the first device will ask the second device to again provide the shared secret to the first device in order to maintain the trusted connection.

Shared-secrets are effective, but as time passes, the risk that the shared secret will be compromised grows (e.g., a third-party might see the code displayed on the first device). Further, even with a shared secret, there is the risk of a brute force attack on the device where an attacker iterates through candidate passkeys until one is accepted.

Security risks aside, in the case of medication therapies, there are social and privacy drawbacks to current pairing techniques. For example, a user may be embarrassed to look at an analyte monitoring device and program a passkey into a medication delivery device in public because it exposes their health condition to strangers. If the devices are visible, that exposes the user to risk because a malicious actor may observe the user and target the user's devices, including in the manner described above. Further, physically reading a shared secret at the first device and entering it at the second device affects the user experience, and can cause a system that is supposed to reduce cognitive burden on a user to be more burdensome. Finally, the first device that provides the passkey must include a user interface sufficient to present the passkey to a user (e.g., a display, light-emitting-diodes, a speaker).

Accordingly, the inventors of this disclosure see a need for systems, methods, and devices that securely facilitate establishing trusted connections among therapy devices that form medication therapy systems.

FIG. 1 is a functional block diagram of a therapy system 100, which includes at least some therapy devices that are configured to establish trusted connections in accordance with one or more embodiments of the disclosure. Therapy system 100 is configured, generally, to monitor a patient and deliver therapeutic amounts of medication to the patient. In one or more embodiments, therapy system 100 includes an analyte monitoring unit 102, medication therapy electronics 104, a remote therapy management system 106, and a medication delivery unit 118.

In one or more embodiments, medication therapy electronics 104 is configured, generally, to analyze therapy related information, provide therapy recommendation to users, and send therapy instructions to medication delivery unit 118. Medication therapy electronics 104 is operatively coupled to medication delivery unit 118, and, in one or more embodiments, medication therapy electronics 104 and medication delivery unit 118 are components of a medication delivery device. In one embodiment, the medication delivery device may be a “smart” medication delivery device, where the medication therapy electronics 104 provides processing power related to the therapy as well as the user experience. For example, the medication delivery device may be an infusion pump system that is configured to automatically deliver medication based on control signals determined at the pump system (e.g., at the medication therapy electronics 104). By way of further example, the medication delivery device may be an injection pen that is configured to provide therapy recommendations based on detected delivery events and/or physiological information about a patient.

In another embodiment, medication therapy electronics 104 may be, or be a component of, a first device and medication delivery unit 118 may be, or be a component of, a second device. The first and second devices may be operatively coupled by a wired or wireless connection. In one embodiment, the connection may be a trusted connection established in accordance with one or more embodiments of the disclosure.

In one or more embodiments, medication therapy electronics 104 is configured to establish a trusted communication link with one or more therapy related devices, including analyte monitoring unit 102 and/or medication delivery unit 118. In one contemplated process for establishing a trusted communication link with analyte monitoring unit 102, medication therapy electronics 104 is configured to provide a candidate shared secret key 116 over an intermediate communication link 114a to analyte monitoring unit 102. In various embodiments, candidate shared secret key 116 may be a value or string of alpha-numeric characters representative of a candidate shared secret to be validated against a shared secret that is unique to analyte monitoring unit 102. In one or more embodiments, shared secrets and candidate shared secrets may be values, strings of alpha-numeric characters, algorithms, or combination thereof. In a contemplated operation of therapy system 100 candidate shared secret key 116 is used, but it is specifically contemplated that there may be multiple pairs of therapy related devices within a therapy system that use different unique shared secrets, candidate shared secrets, and candidate shared secret keys to establish trusted connections. In one embodiment, a shared secret may be unique to therapy system 100 (e.g., a therapy system identifier, algorithm, or combination thereof), and so the same shared secret, candidate shared secret, and candidate shared secret key may be used to establish trusted communication links between some or all of the therapy devices that comprise therapy system 100.

Turning back to the example of FIG. 1, in one or more embodiments, medication therapy electronics 104 provides candidate shared secret key 116 responsive to shared secret parameters that it requests and receives from remote therapy management system 106 over communication network 108. If analyte monitoring unit 102 accepts candidate shared secret key 116, or more specifically, if the analyte monitoring unit 102 verifies the candidate shared secret based, at least in part, on the candidate shared secret key 116, then a trusted communication link is established between analyte monitoring unit 102 and medication therapy electronics 104.

In one embodiment, establishing a trusted communication link includes recording at both devices that intermediate communication link 114a is a trusted communication link. In another embodiment, intermediate communication link 114a is ended and a new communication link 114b is established with trusted communication characteristics (e.g., protocol, encryption, etc.). In various embodiments, the trusted communication link 114b may use the same or different communication equipment (e.g., transceivers configured for BLUETOOTH®, ZIGBEE®, NFC, etc.) as intermediate communication link 114a.

In various embodiments, medication therapy electronics 104 may be configured to communicate over intermediate communication link 114a and trusted communication links, 114b as well as over communication network 108, and may include equipment for several types of communication.

In one or more embodiments, remote therapy management system 106 may execute on one or more computer servers remote from one or more other therapy devices, such as the medication therapy electronics 104, analyte monitoring unit 102, and medication delivery unit 118. The other therapy related devices may be carried with a user, while the remote therapy management system: 106 operates remotely and provides services to devices within the therapy system 100.

In one embodiment, medication therapy electronics 104 sends a shared secret parameters request 112 to remote therapy management system 106. Remote therapy management system 106 receives shared secret parameters request 112 and sends response 110. Response 110 may include, for example, the requested shared secret parameters or a denial message denying the request 112. Medication therapy electronics 104 generates candidate shared secret key 116 responsive to shared secret parameters in the response 110.

Analyte monitoring unit 102 is configured to receive candidate shared secret key 116, and sends an acceptance or rejection message (not shown) over intermediate communication link 114a responsive to candidate shared secret key 116.

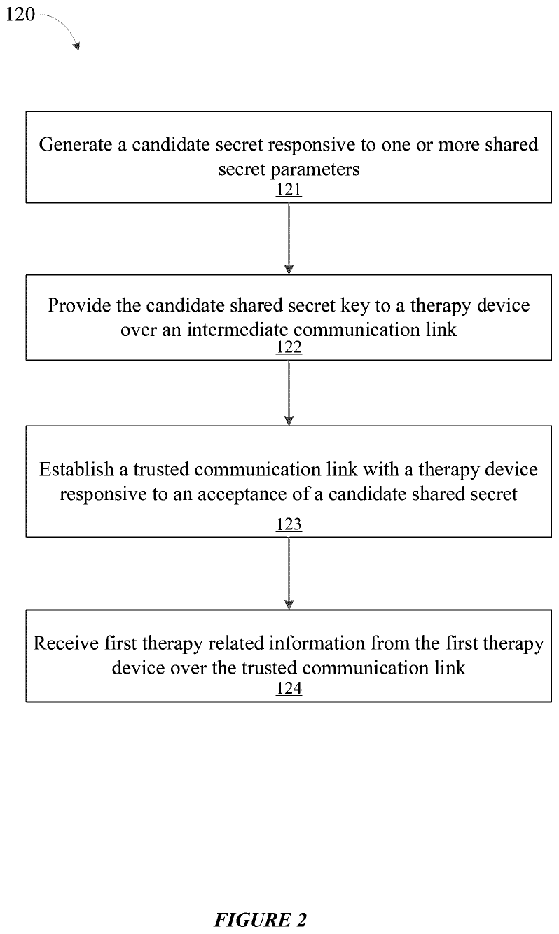

FIG. 2 is a flowchart of a process 120 for establishing a trusted communication among therapy devices, in accordance with one or more embodiments of the disclosure. In operation 121, a candidate shared secret is generated responsive to one or more shared secret parameters. In operation 122, the candidate shared secret key is provided to a therapy device over an intermediate communication link. Candidate shared secret key is representative of one or more candidate shared secrets at a therapy device. In operation 123, a trusted communication link is established with a therapy device responsive to the therapy device accepting a candidate shared secret. In various embodiments and as described more fully herein, a candidate shared secret may be validated based, at least in part, on a candidate shared secret key. The trusted communication link is configured to enable communication of therapy related information from the first therapy device, so, in operation 124, first therapy related information is received from the first therapy device over the trusted communication link.

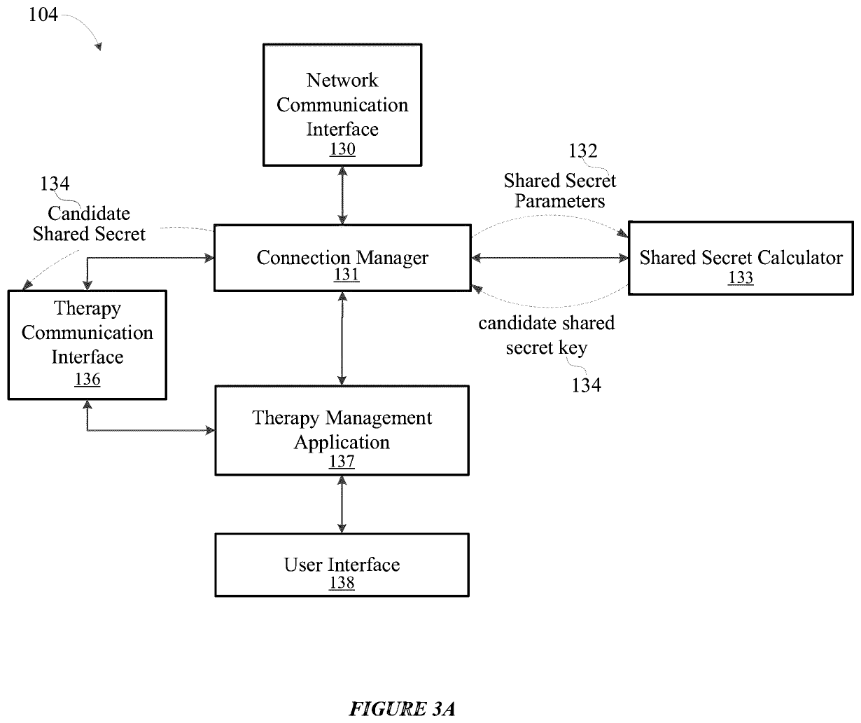

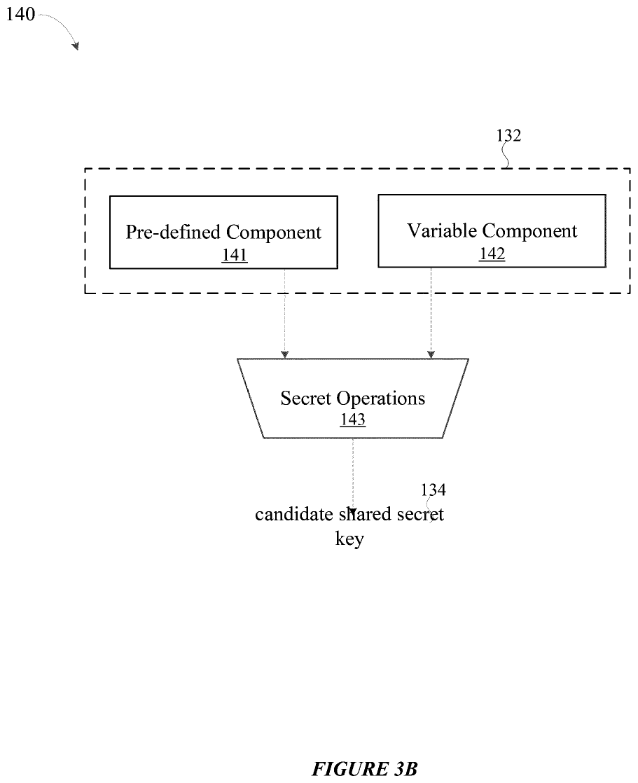

FIG. 3A is a functional block diagram of medication therapy electronics 104, in accordance with one or more embodiments of the disclosure. Medication therapy electronics 104 includes a connection manager 131, a shared secret key calculator 133, a therapy management application 137, and a user interface 138. Medication therapy electronics 104 also includes a network communication interface 130 and a therapy communication interface 136.

Connection manager 131 is configured, generally, to manage establishing one or more communication links with therapy related devices, including, for example, trusted and untrusted (e.g., intermediate) communication links. Connection manager 131 is configured to use network communication interface 130 to send shared secret parameter requests 112 from remote therapy management system 106 (FIG. 1), send one or more shared secret parameters 132 to shared secret key calculator 133 for processing, and to send candidate shared secret keys 134 from shared secret key calculator 133. Connection manager 131 is configured to receive candidate shared secret key 134 and provide candidate shared secret key 134 to a therapy related device (e.g., analyte monitoring unit 102 of FIG. 1) via therapy communication interface 136.

In one or more embodiments, network communication interface 130 may be configured to send requests to, and receive response messages from, remote therapy management system 106 (FIG. 1) over one or more networks, which may include, at least in part, the Internet. By way of example and not limitation, network communication interface 130 may be configured for wireless network communication (e.g., an IEEE 802.11 standard), cellular network communication (e.g., 3G, 4G LTE, 5G, CDMA, GSM etc.), microwave network communication, and combinations thereof.

Therapy communication interface 136 may be configured for personal, local, and/or wide-area network communication with a related therapy device. By way of example, and not limitation, therapy communication interface 136 may be configured for BLUETOOTH communication (including BLUETOOTH Low Energy (BLE), NFC (near field communication), WiFi, ZIGBEE, IrDA (infrared data association), wireless USB (universal serial bus), combinations thereof, and more.

Shared secret key calculator 133 is configured to receive shared secret parameters 132 and provide candidate shared secret key 134. In one or more embodiments, shared secret key calculator 133 is configured to generate candidate shared secret key 134 responsive to shared secret parameters 132 and one or more secret operations. In one embodiment, shared secret key calculator 133 may be pre-configured to perform secret operations, and in another embodiment shared secret key calculator 133 may perform secret operations responsive to secret operation instructions that are included with the shared secret parameters 132.

In various embodiments, one or more shared secret parameters and/or secret operations may be a contemplated shared secret.

FIG. 3B shows a flow chart of a shared secret calculation process 140, in accordance with one or more embodiments of the disclosure. Secret operations 143 are performed responsive to a pre-defined component 141 and a variable component 142. In one embodiment, pre-defined component 141 may be a unique identifier that is associated with the therapy related device with which a trusted communication link is being established. By way of example, unique identifier may be a serial number that is associated with the therapy related device at manufacture or an identifier generated by a remote therapy system. Variable component 142 may be non-static, that is, it may change each time the shared secret calculation process 140 is performed. By way of example, variable component 142 may be time-based—e.g., a time (e.g., “4:28:09”) or calculated using a specific time (e.g., “4-28:09”→4×28×09=1008) If calculated using a specific time, that time may be stored (e.g., in a time stamp format) and sent with the candidate shared secret key 134.

In one embodiment, pre-defined component 141 and variable component 142 are both received by shared secret key calculator 133 (see FIG. 3A) as part of shared secret parameters 132. In another embodiment, shared secret key calculator 133 may be include a variable component calculation module (e.g., a clock circuitry, calculation circuitry, random number generator, etc.) configured to generate variable component 142.

In one or more embodiments, secret operations 143 may be a one-way function configured to obfuscate its input parameters (e.g., pre-defined component 141 and variable component 142). Secret operations 143 may be performed using pre-defined component 141 and variable component 142. In one embodiment, each of pre-defined component 141 and variable component 142 may be a parameter of secret operations 143. In another embodiment, secret operations 143 may use a post-processed parameter that is based, at least in part, on pre-defined component 141 and variable component 142 (e.g., a combination of the two parameters using concatenation, exclusive OR, multiplication, etc.)

In one or more embodiments, secret operations 143 is a hashing function, shared secret parameters 132 is a hash key, and candidate shared secret key 134 is a hash. Shared secret key calculator 133 is configured to generate the hash by hashing the hash key using the hashing function. By using both the pre-defined component 141 and variable component 142, a correct candidate shared secret key 134 is harder to recover illicitly without knowing how the variable component affects secret operations 143.

In one or more embodiments, therapy management application 137 (see FIG. 3A) may be configured, generally, to receive therapy related information (e.g., analyte measurements), and provide one or more of therapy recommendations and therapy instructions. User interface 138 may be configured to provide therapy recommendations generated by therapy management application 137 to a user and receiver user input. In one or more embodiments, user interface 138 may be a graphical user interface presentable at a display operably coupled to medication therapy electronics 104 (see FIG. 1), that is configured to present therapy recommendations and/or therapy related information to a user. By way of example, and not limitation, therapy related information may include notifications, alarms, alerts, combinations thereof, and more. Therapy recommendations may include one or more of changes to medication dosing parameters, recommended bolus doses, recommended correction doses, dietary recommendations (e.g, to eat), physiological recommendations (e.g., sleep or exercise) and more. User input may include approvals, acknowledgements, dismissals, information about therapy relevant events, physiological information, parameters for medication therapies, and more.

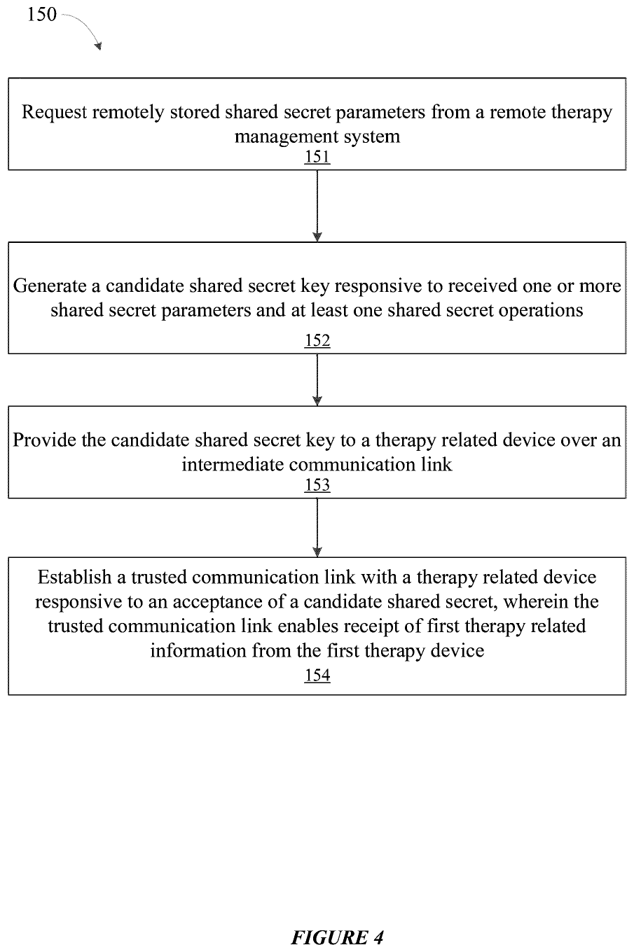

FIG. 4 shows a flowchart of a process 150 for establishing a trusted communication link with another therapy related device, in accordance with one or more embodiments of the disclosure. In one or more embodiments, operations of process 150 may be performed at medication therapy electronics 104 (see FIG. 1), and is one contemplated operation of medication therapy electronics 104. In operation 151, remotely stored shared secret parameters are requested from a remote therapy management system. In one embodiment, shared secret parameters are received in a reply message from the remote therapy system. If the remote therapy management system rejects the request or fails to find the requested shared parameters, a reply message may be received indicating that the request was rejected or the system failed. In operation 152, a candidate shared secret key is generated responsive to received one or more shared secret parameters and at least one shared secret operation. The shared secret parameters may be a unique identifier, and, in one embodiment, may include a variable component as described in this disclosure Shared secret operations may be locally stored or may be remotely stored and received with the shared secret parameters. In operation 153, a candidate shared secret key is provided to the therapy related device. The candidate shared secret key may be provided over an intermediate communication link. By way of example, the intermediate communication link may be an untrusted communication link, a temporary communication link used for establishing a trusted communication link, or a combination thereof. In operation 154, a trusted communication link is established with the therapy related device responsive to an acceptance of a candidate shared secret. The trusted communication link enables receipt of therapy related information from the therapy related device.

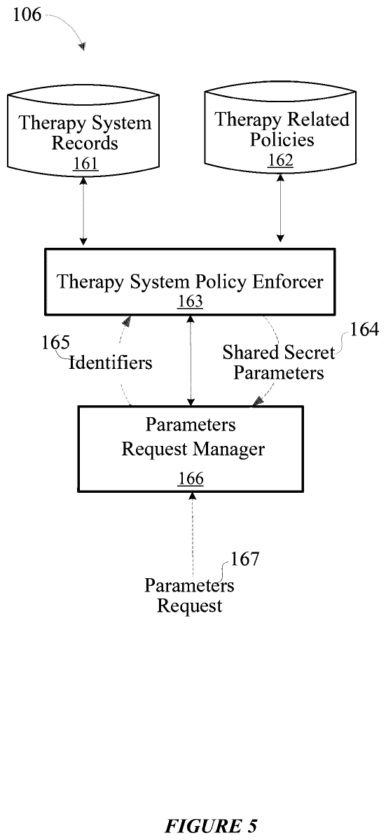

FIG. 5 shows a functional block diagram of remote therapy management system 106, in accordance with one or more embodiments of the disclosure. Remote therapy management system 106 is configured, generally, to manage records related to therapy system 100 (see FIG. 1) and monitor aspects of operation of therapy system 100 and therapeutic delivery of medication more generally. In one or more embodiments, remote therapy management system 106 is configured to monitor and manage therapy system 100 responsive to one or more therapy related policies 162. Therapy related policies 162 may include, for example, restrictions on the number and kinds of therapy related devices that may be part of therapy system 100, as well as physical and operational requirements (e g., security, updates, etc.) for therapy related devices that may be part of therapy system 100.

In one or more embodiments, remote therapy management system 106 may include a parameters request manager 166, a therapy system policy enforcer 163, therapy system records 161, and therapy related policies 162.

In one or more embodiments, parameters request manager 166 may be configured, generally, to manage parameter requests 167 for shared secret parameters received from therapy related devices that are attempting to establish trusted communication links. In one embodiment, parameter request manager 166 may be configured to provide one or more group identifiers 165 to therapy system policy enforcer 163 responsive to receiving a parameter request 167 for shared secret parameters. Group identifiers 165 may be unique identifiers that describe one or more groups of therapy related devices that form medication therapy system. In one embodiment, a group identifier 165 may be a string of digits or characters. In another embodiment, a group identifier 165 may be a patient's name or a string associated with a patient's name (e.g., an anonymizing identifier). In one embodiment, group identifiers 165 may for one or more of the therapy related devices that are attempting to establish a trusted communication link.

Therapy system policy enforcer 163 may be configured, generally, to enforce therapy related policies 162. More particularly, therapy system policy enforcer 163 may be configured to enforce therapy related policies 162 related to forming medication therapy systems from a group of therapy related devices, and more specifically still, to establishing trusted communication links among therapy related devices. So, when a therapy related device requests shared secret parameters, therapy system policy enforcer 163 may be configured to determine whether establishing a trusted communication link would violate one or more therapy related policies 162. For example, if the requesting therapy related device is attempting to establish a trusted communication link with an analyte monitoring unit, therapy system policy enforcer 163 may be configured to reject the request if the therapy system records 161 indicate that the requesting therapy related device already has an active trusted communication link with another, different, analyte monitoring unit. By way of another example, if the requesting therapy related device is attempting to establish a trusted communication link with an analyte monitoring unit, therapy system policy enforcer 163 may be configured to reject the request if the therapy system records 161 indicate that the analyte monitoring unit already has an active trusted communication link with another, different, therapy related device or has reached a maximum number of active trusted communication links with other, different, therapy related devices. By way of yet another example, if the requesting therapy related device is attempting to establish a trusted communication link with an analyte monitoring unit, therapy system policy enforcer 16 may be configured to reject the request if the requesting therapy related device and the other therapy related device are not compatible. For example, therapy related policies 162 may describe lists of compatible types of devices (or, conversely, lists of incomputable devices), and if the requesting therapy related device and the other therapy related device are not compatible then therapy system policy enforcer 16 may be configured to reject the request.

In the contemplated example of FIG. 5, if therapy system policy enforcer 163 determines that the trusted communication link would not violate one or more of therapy related policies 162, then it may retrieve the shared secret parameters responsive to group identifiers 165, therapy system records 161, and the policy determination. Therapy system policy enforcer 163 may provide the shared secret parameters 164 to parameters request manager 166, which in turn may generate and send a reply message to the requesting therapy related device, and the reply message may include shared secret parameters 164. If the request is rejected or fails (e.g., because no relevant therapy system records 161 may be found responsive to group identifiers 165), then the reply message may indicate the rejection or failure.

Therapy system records 161 may include records stored in a database that are related to one or more medication therapy systems. Therapy system records 161 may describe therapy related devices that form a medication therapy system, including identifiers, type of devices, patient information, insurance information, provider information, and more.

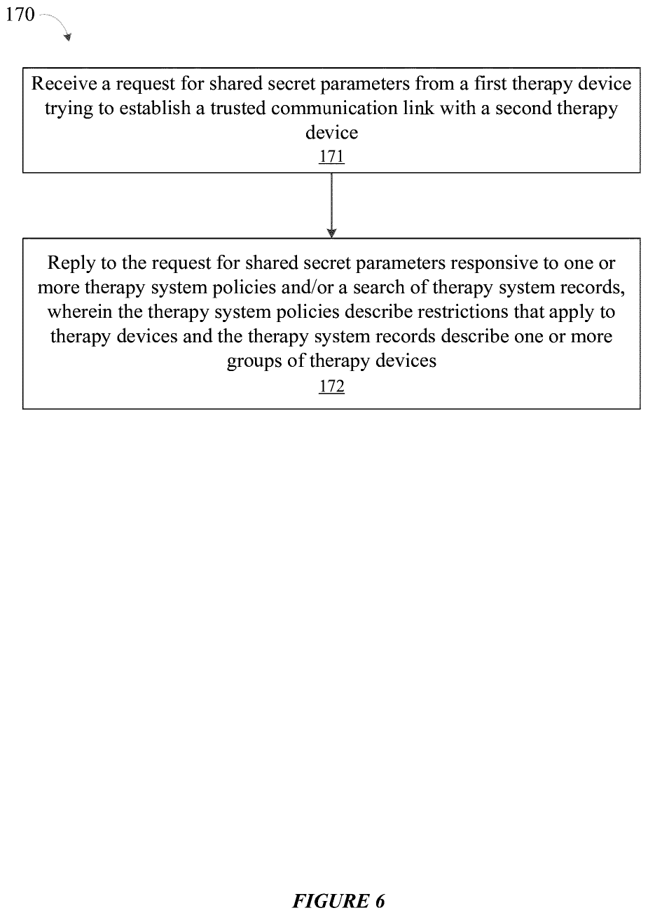

FIG. 6 is a flowchart of a process 170 for enforcing policies for establishing trusted communication links among therapy devices, in accordance with one or more embodiments of the disclosure. In one or more embodiments, operations of process 170 may be performed at remote therapy management system 106, and is one contemplated operation of remote therapy management system 106. In operation 171, a request for shared secret parameters is received. The request may be received from a first therapy device that is attempting to establish a trusted communication link with a second therapy device. In operation 172, a reply message is sent responsive to the request for shared secret parameters. The contents of the reply message may be selected responsive to one or more therapy system policies and/or searches of therapy system records, and may include, for example, shared secret parameters or a denial message. The therapy system policies and therapy system records may be managed at one or more databases. Therapy system policies may describe restrictions that apply to therapy devices and groups of therapy devices. Therapy system records may describe individual therapy devices and groups of therapy devices that form medication therapy systems.

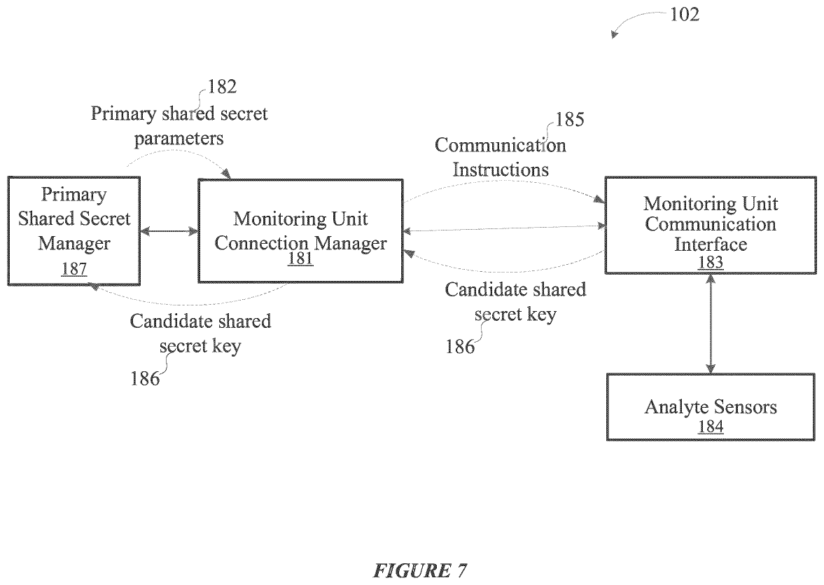

FIG. 7 is a functional block diagram of analyte monitoring unit 102, in accordance with one or more embodiments of the disclosure. Analyte monitoring unit 102 is configured, generally, to measure analyte levels in blood samples and send the measurements to therapy related devices that, together with analyte monitoring unit 102, may be part of a medication therapy systems. One or more embodiments of analyte monitoring unit 102 may include a monitoring unit connection manager 181, a monitoring unit communication interface 183, and analyte sensors 184.

Monitoring unit communication interface 183 is configured, generally, to send and receive messages to and from other therapy related devices, for example, over trusted and/or untrusted communication links. In one or more embodiments, messages sent by monitoring unit communication interface 183 may include one or more of analyte measurement information received from analyte sensors 184, and status information about analyte monitoring unit 102 (e.g., battery life, component health, etc.). Monitoring unit communication interface 183 may also be configured to send and receive messages related to establishing trusted communication links with other therapy related devices.

Monitoring unit connection manager 181 is configured, generally, to manage establishment of trusted communication links with other therapy related devices. Monitoring unit connection manager 181 may include or have access to primary shared secrets 182 stored at analyte monitoring unit 102. In a contemplated operation of analyte monitoring unit 102, when a therapy device attempts to establish a trusted communication link with analyte monitoring unit 102, monitoring unit connection manager 181 is configured to validate a candidate shared secret responsive to candidate shared secret key 186 and primary shared secrets 182.

In one or more embodiments, primary shared secret manager 187 is configured to generate and provide primary shared secret parameters 182 to monitoring unit connection manager 181. In one embodiment, primary shard secret manager 187 stores one or more primary shared secret parameters that it provides to connection manager 181. In one contemplated operation, the primary shared secret parameters may include a unique identifier (e.g., a string of alpha numeric characters), a decryption key, and an encrypted string (e.g., cyphertext) that was generated based, at least in part, on the unique identifier, an encryption algorithm corresponding to the decryption algorithm, and an encryption key. Connection manager 181 may decrypt the encrypted string using the decryption algorithm and the candidate shared secret key 186. In a contemplated operation, if the decrypted string matches the unique identifier than that is indicative that a candidate shared secret matches a primary shared secret of the analyte monitoring unit 102. The primary shared secret may be a unique identifier, a hash function, or both.

In another embodiment, primary shared secret parameters 182 may be a primary shared secret key generated by primary shared secret manager 187. Primary shared secret manager 187 may perform secret operations with a unique identifier to generate a primary key, and provide the primary key to connection manager 181 to compare the primary key to candidate shared secret key 186. In a case where candidate shared secret key 186 matches the primary key then that is indicative that the primary secret operations and primary shared secret parameters are the same as the ones used to generate candidate shared secret key 186. For example, the secret operations performed at primary shared secret manager 187 may be the same hashing function or other one-way function performed at shared secret key calculator 133 (see FIG. 3A). In a case where candidate shared secret key 186 does not match a primary key, then any of the secret operations and shared secret parameters may be different than those used to generate candidate shared secret key 186.

Any suitable technique known by one of ordinary skill in the art may be used by monitoring unit connection manger 181 to perform comparisons described herein. For example, a symbol-to-symbol comparison, bit-to-bit comparison, converting to an integer (e.g., if they are strings) and comparing integer values, and the like.

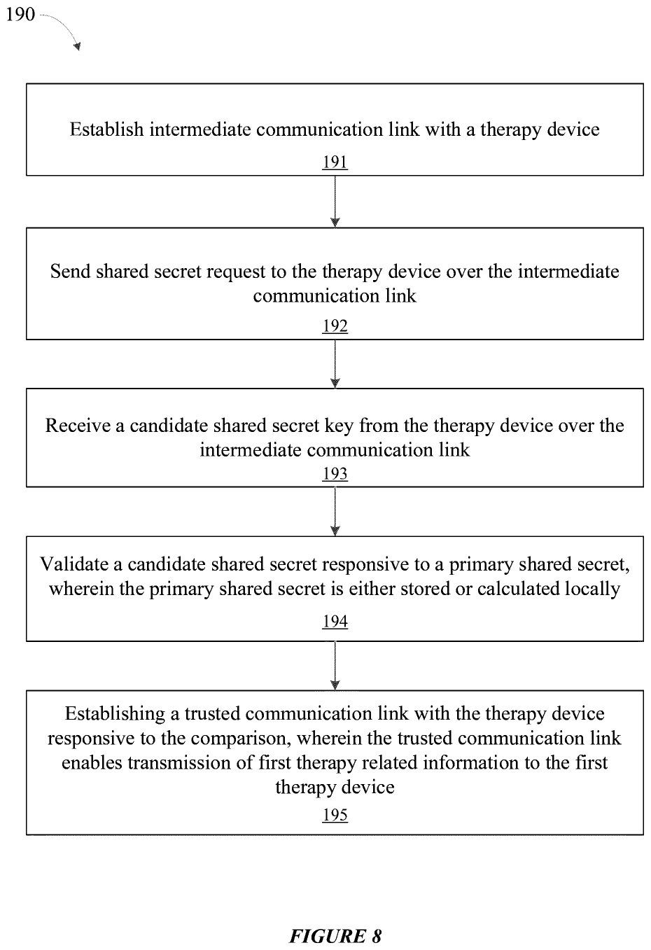

FIG. 8 is a flowchart of a process 190 for establishing trusted communication links with a therapy device, in accordance with one or more embodiments of the disclosure. In one or more embodiments, operations of process 190 may be performed at analyte monitoring unit 102 (see FIG. 1 and/or FIG. 7). In operation 191, an intermediate communication link is established with a therapy device that is attempting to establish a trusted communication link. In operation 192, a request for a shared secret is sent to the therapy device over the intermediate communication link. In operation 193, a candidate shared secret key is received from the therapy device over the intermediate communication link. In one embodiment, a countdown is implemented and if the candidate shared secret key is not provided within a certain amount of time, the request “times out” and a candidate shared secret is automatically rejected. This avoids a problem whereby the requesting therapy device is waiting in an active intermediate mode, which may happen if the requesting therapy device fails to receive shared secret parameters from a remote therapy management system. In operation 194, a candidate shared secret is validated responsive to a primary shared secret. In various embodiments, the primary shared secret or values indicative of it may be stored or calculated locally. In operation 195, a trusted communication link is established with the requesting therapy device responsive to the comparison. For example, if a candidate shared secret matches a primary shared secret then an acceptance message is provided to the requesting therapy device. If a candidate shared secret does not match a primary shared secret then a rejection message is provided to a requesting therapy device. Once the trusted communication link is established, therapy related information may be sent between therapy devices. For example, analyte monitoring unit 102 may send analyte measurement data 326 to a therapy related device that includes without limitation medication therapy electronics 104.

One or more embodiments relate, generally, to a process for establishing trusted connections that may be performed by therapy devices using shared secrets stored or determined locally, and systems and devices for accomplishing the same.

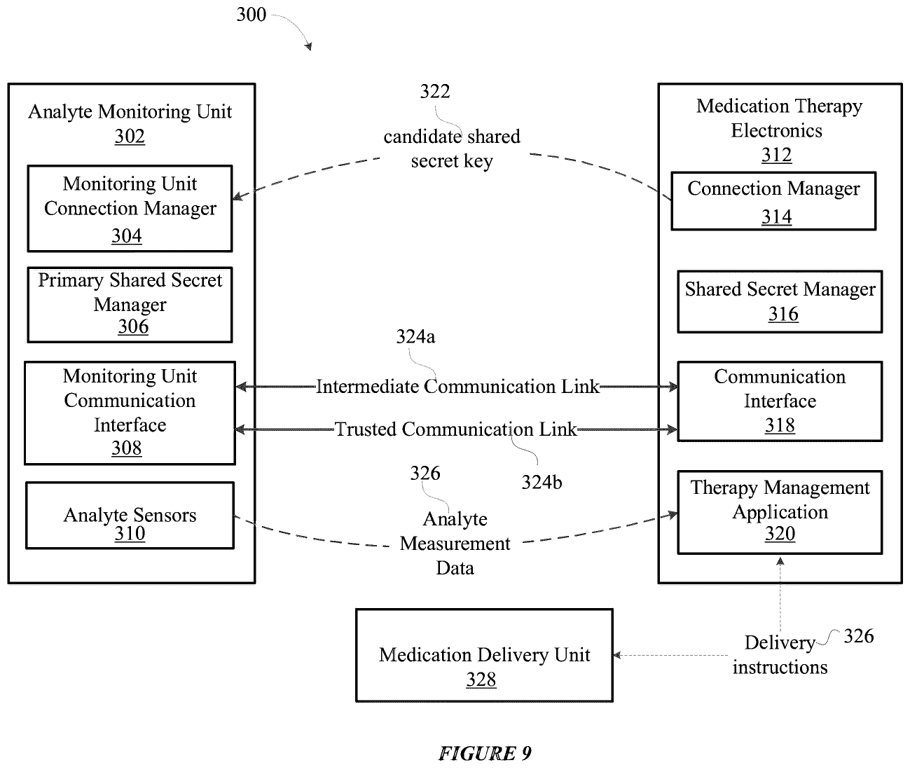

FIG. 9 is a functional block diagram of therapy system 300 that includes at least some therapy devices that are configured to establish trusted connections in accordance with one or more embodiments of the disclosure. Therapy system 300 is configured, generally, to monitor a patient and deliver therapeutic amounts of medication to the patient. In one or more embodiments, therapy system 300 includes an analyte monitoring unit 302, medication therapy electronics 312, and a medication delivery unit 328.

Notably, a remote therapy system is not shown as part of therapy system 300 because, in one or more embodiments, medication therapy electronics 312 is configured to store and/or calculate the shared secret locally. Nevertheless, one of ordinary skill in the art would understand that therapy system 300 may include remote therapy management elements.

In one or more embodiments, medication therapy electronics 312 includes a connection manager 314, a shared secret manager 316, a communication interface 318, and a therapy management application 320. In one embodiment, shared secret manager 316 is configured to retrieve a locally stored candidate shared secret key 322 responsive to a request to provide a shared secret from analyte monitoring unit 302, and connection manager 314 is configured to provide candidate shared secret key 322 to analyte monitoring unit 302. Candidate shared secret key 322 may be, for example, a hash of a unique identifier associated with analyte monitoring unit 302.

In one or more embodiments, shared secret manager 316 is configured to determine candidate shared secret key 322. In one embodiment, a secret and one or more secret operations are stored at medication therapy electronics 312. By way of example, the secret may be a hash key and the secret operations may be a hashing function Shared secret manager 316 determines candidate shared secret key 322 by performing secret operations using secrets as a parameter.

In one or more embodiments, a secret may be static, for example, it may be a unique identifier associated with analyte monitoring unit 302. In one or more embodiments, the secret operations may be configured to use at least two parameters: a static component (e g., a unique identifier) and a variable component (e.g., a time), such that the candidate shared secret key 322 is different for different variable components. In yet another embodiment, the secret may be a combination of a unique identifier and a variable component.

In one or more embodiments, analyte monitoring unit 302 may include monitoring unit connection manager 304, primary shared secret manager 306, monitoring unit communication interface 308, and analyte sensors 310. Monitoring unit connection manager 304 is configured to validate a candidate shared secret based, at least in part, on candidate shared secret key 322 responsive to a primary shared secret provided by primary shared secret manager 306, for example, by comparing candidate shared secret key 322 to a primary shared secret or a primary shared secret key generated using a primary shared secret.

In one embodiment, analyte monitoring unit 302 may store a primary shared secret. In another embodiment, primary secret parameters and secret operations may be stored at analyte monitoring unit 302, and primary shared secret manager 306 may be configured to determine a primary shared secret or a primary shared secret key, at least in part, using secret operations. In the example of FIG. 8, if secret operations and a secret key used by primary shared secret manager 306 are the same as secret operation(s) and a secret key(s) used by shared secret manager 316 of medication therapy electronics 312 to generate candidate shared secret key 322, then primary shared secret key will match candidate shared secret key 322. If a candidate shared secret is validated at least in part using candidate shared secret key 322, then analyte monitoring unit 302 is configured to send an acceptance message to medication therapy electronics 312, and the two devices may establish trusted communication link 324b.

Notably, candidate shared secret key 322 may also include a variable component if used to generate it, for example, a time stamp. Primary shared secret manager 306 may generate a primary shared secret or primary shared secret key responsive to a variable component, primary secret operations, and primary shared secret parameters.

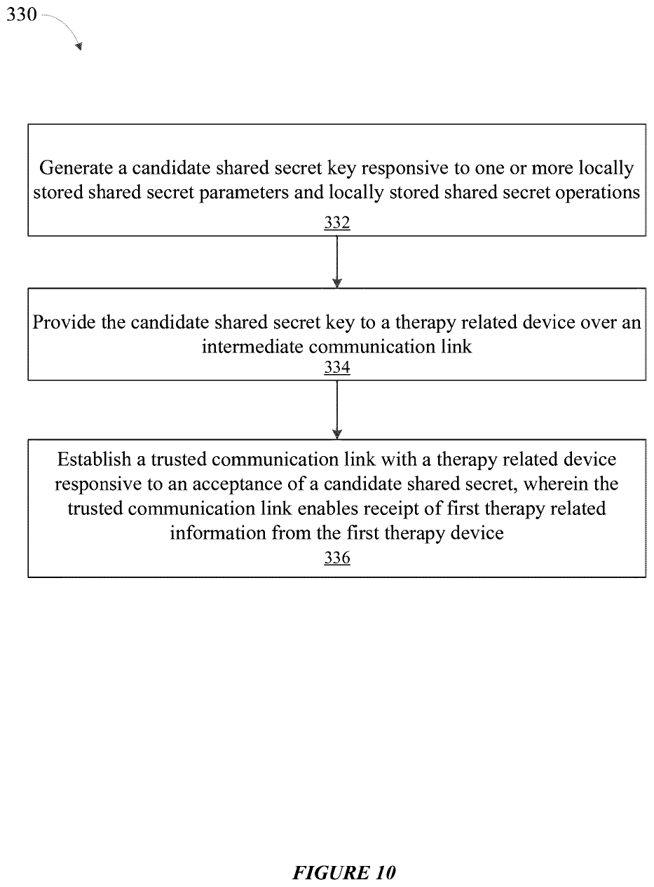

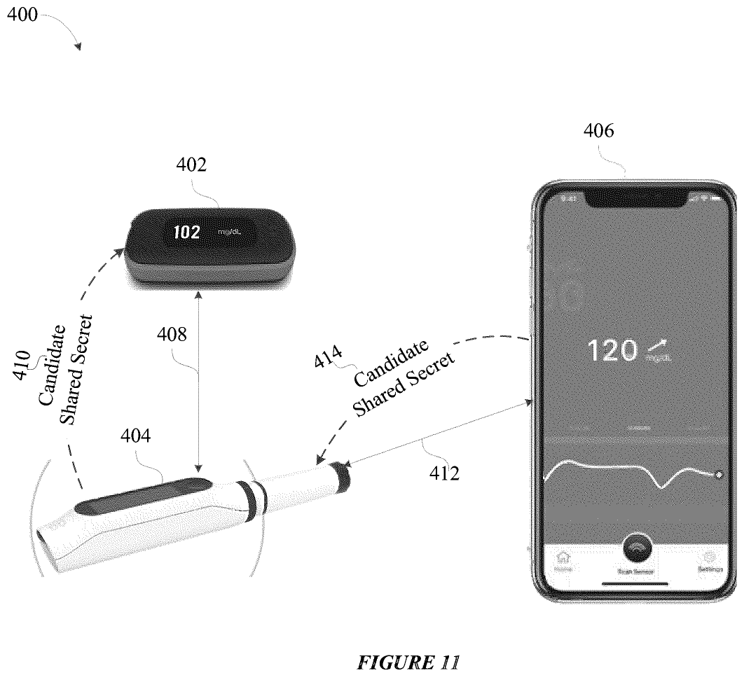

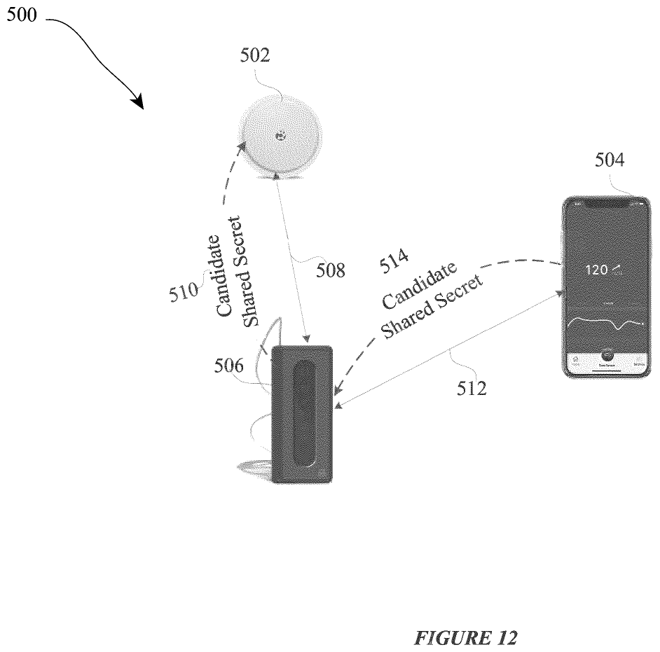

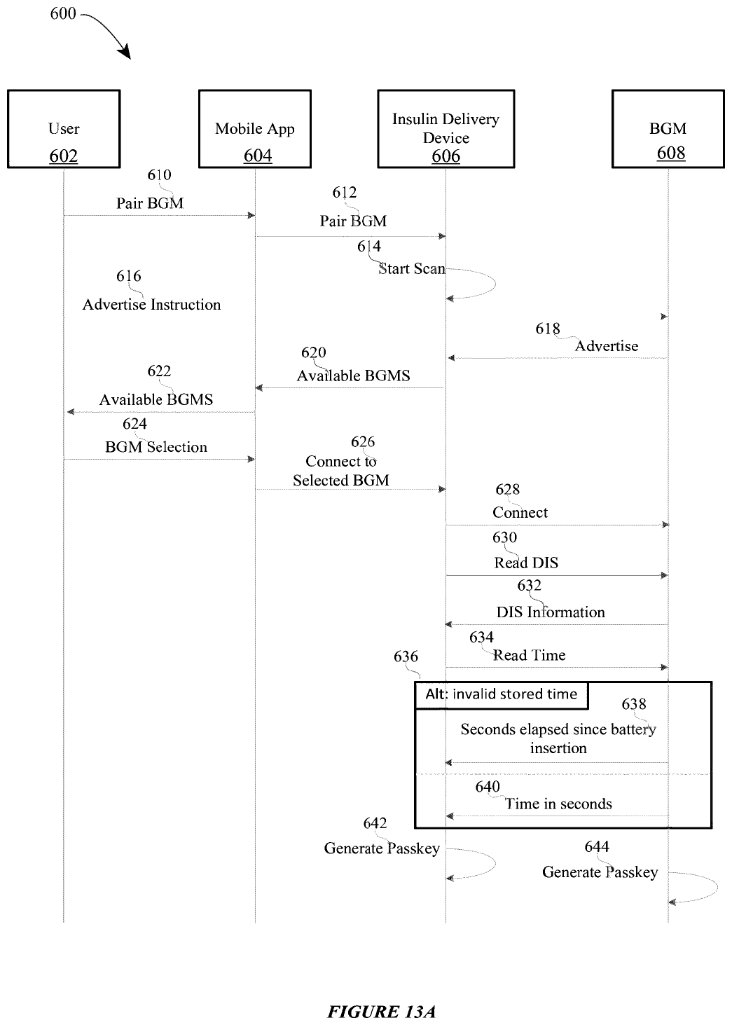

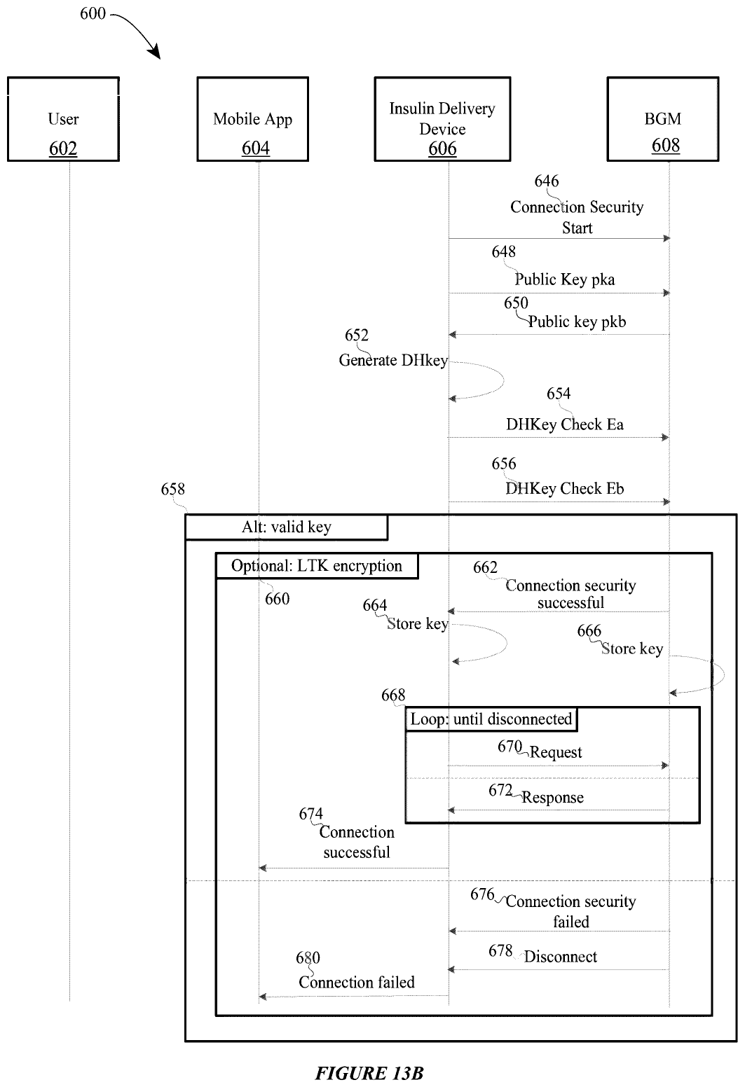

FIG. 10 is a flowchart of a process 330 for establishing a trusted communication link with another therapy related device, in accordance with one or more embodiments of the disclosure. In one or more embodiments, operations of process 330 may be performed at medication therapy electronics 312, and is one contemplated operation of medication therapy electronics 312. In operation 332, a candidate shared secret key is generated responsive to one or more locally stored shared secret parameters and locally stored shared secret operations. In various embodiments, the shared secret parameters may include static components, variable components, and/or secret operations. In operation 334, a candidate shared secret key is provided to a therapy related device over an intermediate communication link 324a. In operation 336, a trusted communication link is established with the therapy related device responsive to an acceptance of a candidate shared secret. Acceptance may be communicated responsive to validating a candidate share secret using a candidate shared secret key. In one or more embodiments, a trusted communication link is configured to enable receipt of therapy related information from the therapy device.

One or more embodiments of the disclosure relate to insulin therapy systems configured to implement pairing techniques described in this disclosure.

Diabetes mellitus is a chronic metabolic disorder caused by the inability of a person's pancreas to produce sufficient amounts of the hormone insulin such that the person's metabolism is unable to provide for the proper absorption of sugar and starch. This failure leads to hyperglycemia, i.e., the presence of an excessive amount of glucose within the blood plasma. Persistent hyperglycemia has been associated with a variety of serious symptoms and life threatening long-term complications such as dehydration, ketoacidosis, diabetic coma, cardiovascular diseases, chronic renal failure, retinal damage and nerve damages with the risk of amputation of extremities. Because healing is not yet possible, a permanent therapy is necessary which provides constant glycemic control in order to constantly maintain the level of blood analyte within normal limits. Such glycemic control is achieved by regularly supplying external drugs to the body of the patient to thereby reduce the elevated levels of blood analyte.

An external biologically effective drug (e g., insulin or its analog) is commonly administered by means of daily injections. In some cases, multiple, daily injections (MDI) of a mixture of rapid- and long-acting insulin via a reusable transdermal liquid dosing device (commonly referred to as an “insulin pen”) or a hypodermic syringe. The injections are typically administered by a person with diabetes (PWD), and so requires self-monitoring of blood glucose and the self-administration of insulin. The PWD that manages their care using MDI often plans insulin injections for each day, in advance, based on basal insulin requirement as well as external factors such as meals, exercise, sleep, etc. A typical dosing plan will include the time of day for an injection, the type of insulin (e.g., fast acting, long acting, a mixture of fast acting and long acting, etc.), and amount of insulin for each dose. In addition, PWDs will self-monitor their blood glucose and self-administer “bolus” dose(s) of rapid-acting insulin if their blood glucose is too high or consume carbohydrates (or sometimes administer glycogen) if their blood glucose is too low.

The “correct” insulin dose is a function of the level of glucose in the blood, physiological factors such as a person's insulin sensitivity, and lifestyle factors such as meals (e.g., recently consumed carbohydrates that have yet to be metabolized into glucose and absorbed into the blood). Moreover, even with careful planning and self-monitoring, a PWD may skip doses, double dose, and dose the wrong amount and/or type of insulin. Insufficient insulin can result in hyperglycemia, and too much insulin can result in hypoglycemia, which can result in clumsiness, trouble talking, confusion, loss of consciousness, seizures, or death. Accordingly, PWDs face a considerable cognitive burden in determining appropriate doses of insulin.

In order to assist with self-treatment, some insulin therapy devices (e g., blood glucose meters, continuous glucose meters, insulin pumps, insulin pens etc.) are equipped with insulin therapy applications that assist user with making appropriate therapy decisions while minimizing the burdens of data entry, mental calculations, procedures, etc.). For example, insulin bolus calculators may be used that have the user input an estimate (e.g., numerical estimate) of the quantity of carbohydrates consumed or about to be consumed (or additionally or alternatively protein, fat, or other meal data) and the bolus calculator outputs a recommended size for the insulin bolus dosage. Although bolus calculators remove some of the mental calculations that need to be made by the user in determining an appropriate insulin bolus dosage, bolus calculators still burden the user with the mental task of evaluating the constituents of their meal, may require the use of a secondary device, and often require manual entry of data.

Automated insulin delivery may also be used to relieve some of the burdens of self-treatment. A glucose monitoring unit measures levels of glucose in blood samples and sends at least some of the measurements to a therapy application that is monitoring patient's response to insulin therapy. The therapy application determines insulin dosing (e.g., amount, timing, rates of insulin, etc.) dosing to account for a patient's basal insulin needs as well as to account for physiological events such as carbohydrate intake, exercise, sleep, medication intake (other than insulin), and more. The therapy application uses glucose levels received from the glucose monitoring unit as a control variable to determine insulin dosing instructions that it then sends to an insulin delivery device, such an infusion pump. The operation of the insulin delivery device is configured by the insulin dosing instructions and the insulin delivery device will then deliver insulin according to the new configuration. Insulin therapy systems that implement automated insulin delivery may operate in “open loop” modes and “closed loop” modes. By way of example, in an open loop mode, the insulin therapy system may involve some user interaction before sending the insulin delivery instructions 326 to the insulin delivery device (e.g., a user acceptance of change). In a closed loop mode, the therapy application may determine and automatically send the insulin delivery instructions 326 to the insulin delivery device without user interaction.

FIG. 11 shows an insulin therapy system 400 configured for pairing in accordance with one or more embodiments of the disclosure. Insulin therapy system 400 includes a blood glucose meter (BGM) 402, an insulin injection pen 404, and a mobile computing device 406 with an insulin therapy application executing thereon.

In one or more embodiments, insulin injection pen 404 and BGM 402 are configured for pairing in accordance with the embodiments for establishing a trusted communication link described in this disclosure. In one embodiment, a shared secret is locally stored at BGM 402, and upon request, insulin injection pen 404 is configured to provide a candidate shared secret to BGM 402 in order to pair with BGM 402 and start receiving glucose level measurement data from BGM 402. If insulin injection pen 404 fails to provide candidate shared secret 410 or BGM 402 does not validate candidate shared secret 410 (e.g., using its stored primary shared secret, or using a shared secret key and secret operations), pairing will fail and trusted communication link 408 will not be established.

In one or more embodiments, mobile computer device 406 and insulin injection pen 404 are configured for pairing in accordance with the embodiments for establishing a trusted communication link described in this disclosure. In one embodiment, a shared secret is locally stored at insulin injection pen 404, and upon request, mobile computing device 406 is configured to provide a candidate shared secret 414 in order to pair with insulin injection pen 404 and start receiving insulin dosing event information from insulin injection pen 404. If mobile computing device 406 fails to provide candidate shared secret 414 or if insulin injection pen 404 does not validate candidate shared secret 410 against its stored primary shared secret, pairing will fail and trusted communication link 412 will not be established.

FIG. 12 shows an automated insulin therapy system 500 configured for pairing in accordance with one or more embodiments of the disclosure. Automated insulin therapy system 500 includes a continuous glucose monitor (CGM) 502, an insulin infusion pump 506, and a mobile computing device 504 with an insulin therapy application executing thereon. Automated insulin therapy system 500 may be configured for open loop and closed loop delivery of insulin to a patient.

In one or more embodiments, insulin infusion pump 506 and CGM 502 are configured for pairing in accordance with the embodiments for establishing a trusted communication link described in this disclosure. In one embodiment, a shared secret is locally stored at CGM 502, and upon request, insulin infusion pump 506 is configured to provide a candidate shared secret 510 to CGM 502 in order to pair with CGM 502 and start receiving glucose level measurement data from CGM 502. If insulin infusion pump 506 fails to provide candidate shared secret 510 or CGM 502 does not validate candidate shared secret 510 (e.g, using its stored primary shared secret, or using a shared secret key and secret operations), pairing will fail and trusted communication link 508 will not be established.

By using the process for establishing trusted communication links, mobile computing devices and infusion pumps that are not part of automated insulin therapy system 500 may be restricted from receiving blood glucose level measurement data from CGM 502.

In one or more embodiments, mobile computer device 504 and insulin infusion pump 506 are configured for pairing in accordance with the embodiments for establishing a trusted communication link described in this disclosure. In one embodiment, a shared secret is locally stored at insulin infusion pump 506, and upon request, mobile computing device 504 is configured to provide a candidate shared secret 514 in order to pair with insulin infusion pump 506 and start receiving insulin dosing event information from insulin infusion pump 506. If mobile computing device 504 fails to provide candidate shared secret 514 or if insulin infusion pump 506 does not validate candidate shared secret 514 against its stored primary shared secret, pairing will fail and trusted communication link 512 will not be established. By using the process for establishing trusted communication links, mobile computing devices that are not part of automated insulin therapy system 500 may be restricted from receiving insulin dosing event information from insulin infusion pump 506 and sending insulin dosing instructions to insulin infusion pump 506.

FIGS. 13A and 13B show a sequence of operations that are part of a pairing process 600 performed by a BGM 608, mobile application 604, and insulin delivery device 606 (e.g., an insulin injection pen, medication delivery accessory such as a pen cap, an insulin infusion pump, etc.) that together form, at least in part, an insulin therapy system, in accordance with one or more embodiments of the disclosure. One or more operations of pairing process 600 may be performed by a user 602 of the insulin therapy system. In one or more embodiments, operations indicated as performed by a user may be performed by a user action to activate GUI elements, actuate buttons, speaking voice commands, physically performing pre-defined motions (e.g., waiving a device near another device), etc. Performing the user action(s) initiates some recognition of the action and therefore the operation(s) at a device that is acted on. However, this is not intended to limit this disclosure to requiring a specific user action(s). For example, one or more embodiments may include a recognition of a user action or what is assumed to be a user action, but not the user action itself.

In operation 610, user 602 provides BGM pairing instruction to mobile application 604. In operation 612, mobile application 604 provides a BGM pairing instruction to insulin delivery device 606 responsive to the BGM pairing instruction received in operation 610. Pairing operations between insulin delivery device 606 and BGM 608 are initiated by insulin delivery device 606 and BOM 608 “discovering” each other. In operation 614, insulin delivery device 606 enters a scanning mode responsive to receiving the BGM pairing instruction in operation 612, and scans for advertising messages. In operation 616, user 602 provides an advertise instruction to BGM 608, for example, but actuating a button for some pre-defined period of time. In operation 618, BGM enters and advertising mode and sends advertisement messages that may be received by nearby devices. In operation 620, insulin delivery device 606 sends to mobile application 604 an available BGM list comprising one or more identifiers for available BGM devices (e.g., unique BGM devices for which advertisement messages have been received in the most recent scan mode period), including an identifier for BGM 608. In operation 622, mobile application 604 presents the available BGM list at a display for user 602. In operation 624, user 602 provides a BGM selection to mobile application 604, the BGM selection uniquely identifying one of the BGM devices listed in the BGM list. In operation 626, responsive to the selection, mobile application 604 provides a connection instruction to insulin delivery device 606 that, at least in part, identifies the selected BGM device-here, BGM 608.

In operation 628, insulin delivery device 606 provides a connection request to BGM 608 responsive to receiving the connection instruction in operation 626. In operation 630, insulin delivery device 606 provides a read device information service (DIS) request (or some other request for identifying information, without limitation) to BGM 608. In operation 632, BGM 608 provides DIS information (or other identifying information, without limitation) to insulin delivery device 606 responsive to the read DIS request in operation 630.

In operation 634, insulin delivery device 606 provides a read time request to BGM 608. In one embodiment, BGM 608 provides stored time information (e.g., in seconds) to insulin delivery device 606 in operation 640, responsive to the read time request in operation 634. In another embodiment, BGM 608 may determine in operation 636 whether the stored time is usable or unusable (e.g., because it is incorrect). Responsive to a determination that the stored time is usable, BGM 608 may provide the time in seconds in operation 640. Responsive to a determination that the stored time is not usable, BGM 608 may provide the seconds elapsed since last battery insertion in operation 638.

In operation 642, insulin delivery device 606 generates a passkey responsive to the time information received in operations 640 (or in operation 638) and the DIS information received in operation 632. At about the same time as operation 642, in operation 644 BGM 608 generates a passkey responsive to the time information sent in operation 640 and the DIS information sent in operation 632.

In operation 646, insulin delivery device 606 provides a connection security start request to BGM 608 to attempt to establish a trusted communication link between insulin delivery device 606 and BGM 608. In operation 648, insulin delivery device 606 provides an authentication request that includes a public key “pka” to BGM 608. In operation 650, BGM 608 provides a public key “pkb” to BGM 608 responsive to receiving the authentication request in operation 648. Up receipt of “pkb,” insulin delivery device 606 and BGM 608 may send and receive messages using public key encryption.

In operation 652, insulin delivery device 606 generates DHkey, which is a candidate shared secret, in accordance with tone or more processes for generating candidate shared secrets of this disclosure. In operations 654 and 656, insulin delivery device 606 provides a verification request to BGM 608 that includes DHKey encrypted using the public encryption keys “pka” and “pkb.”

In operation 658, BGM 608 determines whether DHkey is a valid key or an invalid key in accordance with one or more processes for validating candidate shared secrets of this disclosure Responsive to determining that DHkey is an invalid key, in operation 676 BGM 608 provides a connection security failure response to insulin delivery device 606, and in operation 678 BGM 608 sends a disconnect message to insulin delivery device 606 to end the untrusted communication link, and terminates the link locally. In operation 680, insulin delivery device 606 sends a connection failure message to mobile application 604, which, in one or more embodiments, presents a failure message to user 602.

Responsive to determining that DHkey is a valid key, in operation 662, BGM 608 sends a connection successful message to insulin delivery device 606 and stores the DHkey in operation 666. In operation 664, insulin delivery device 606 stores the DHkey responsive to receiving the connection successful message provided in operation 662. In operation 674, insulin delivery device 606 provides a connection successful message to mobile application 604.

In one or more embodiments, insulin delivery device 606 and BGM 608 may be configured to continuously perform a truncated authentication process 668 while the trusted communication link is active. In operation 670, insulin delivery device 606 may provide a request to continue the trusted communication link to BGM 608. The request may include DHkey or a new key that is the result of modifying DHkey according to some pre-agreed operations. In operation 672, BGM 608 may provide a response message indicating that the truncated authentication was successful, for example, responsive to validating the DHkey or new key received from insulin delivery device 606 in operation 670.

While one-way sharing of candidate shared secrets has been generally described in this disclosure, one of ordinary skill in the art would understand that two-way sharing of candidate shared secrets is within the scope of the disclosure. When two therapy devices attempt to pair, each therapy device may require the other therapy device to provide a shared secret in order to establish a trusted communication link. In one or more embodiments that implement two-way sharing, each therapy related device may include means for requesting, generating, and/or retrieving a candidate shared secret to provide to the other therapy related device. Similarly, each therapy related device may include means for validating a received candidate shared secret.

One or more of shared secrets, shared secret parameters, secret keys, and secrete operations, may be stored on therapy devices at manufacture or during an initial setup. For example, before an analyte monitoring unit is provided to a patient, the serial number may be used to generate a shared secret that is stored at the monitoring unit and provided either to the patient's specially configured mobile computing device or a remote therapy management system. Alternatively or in addition, the operations used to generate the shared secret may be provided to the patient's specially configured mobile computing device or the remote therapy management system along with the parameters used to generate the shared secret.

Information and signals described herein may be represented using any of a variety of different technologies and techniques. For example, data, instructions, commands, information, signals, bits, symbols, and chips that may be referenced throughout the description may be represented by voltages, currents, electromagnetic waves, magnetic fields or particles, optical fields or particles, or any combination thereof. Some drawings may illustrate signals as a single signal for clarity of presentation and description. It should be understood by a person of ordinary skill in the art that the signal may represent a bus of signals, wherein the bus may have a variety of bit widths and the disclosure may be implemented on any number of data signals including a single data signal.

It should be understood that any reference to an element herein using a designation such as “first,” “second,” and so forth does not limit the quantity or order of those elements, unless such limitation is explicitly stated. Rather, these designations are used herein as a convenient method of distinguishing between two or more elements or instances of an element. Thus, a reference to first and second elements does not mean that only two elements can be employed or that the first element must precede the second element in some manner. Also, unless stated otherwise a set of elements may comprise one or more elements. Likewise, sometimes elements referred to in the singular form may also include one or more instances of the element.

Many of the functional descriptions in this specification may be illustrated, described or labeled as modules, threads, steps, or other segregations of programming code, including firmware, in order to more particularly emphasize their implementation independence. Modules may be at least partially implemented in hardware, in one form or another. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices, or the like.

Modules may also be implemented using software or firmware, stored on a physical storage device (e.g., a computer readable storage medium), in memory, or a combination thereof for execution by various types of processors.

An identified module of executable code may, for instance, comprise one or more physical or logical blocks of computer instructions, which may, for instance, be organized as a thread, object, procedure, or function. Nevertheless, the executable of an identified module need not be physically located together, but may comprise disparate instructions stored in different locations which, when joined logically together, comprise the module and achieve the stated purpose for the module.

Indeed, a module of executable code may be a single instruction, or many instructions, and may even be distributed over several different code segments, among different programs, and across several storage or memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the software portions are stored on one or more physical devices, which are referred to herein as computer readable media.

In some embodiments, the software portions are stored in a non-transitory state such that the software portions, or representations thereof, persist in the same physical location for a period of time. Additionally, in some embodiments, the software portions are stored on one or more non-transitory storage devices, which include hardware elements capable of storing non-transitory states and/or signals representative of the software portions, even though other portions of the non-transitory storage devices may be capable of altering and/or transmitting the signals Examples of non-transitory storage devices are flash memory and random-access-memory (RAM). Another example of a non-transitory storage device includes a read-only memory (ROM) which can store signals and/or states representative of the software portions for a period of time. However, the ability to store the signals and/or states is not diminished by further functionality of transmitting signals that are the same as or representative of the stored signals and/or states. For example, a processor may access the ROM to obtain signals that are representative of the stored signals and/or states in order to execute the corresponding software instructions.

Some embodiments of the disclosure include or are described as implementing a server. A server is a computer program that provides functionality or services to other programs, commonly called clients. While a server is a computer program or process (i.e., executing program), the term may also be used to refer to a computer running one or more server programs, and so, unless otherwise indicated, the use of the term server in this description is intended to cover both situations. Further, the term computer is intended to cover a single machine, several machines (e.g., a server farm), as well as virtual clusters of computers that emulate one or more hardware elements including without limitation central processing units, graphics processing units, local and system memory, memory storage (e.g., hard disks and solid state drives), operating systems, and networking equipment.

Any characterization in this disclosure of something as ‘typical,’ ‘conventional,’ or ‘known’ does not necessarily mean that it is disclosed in the prior art or that the discussed aspects are appreciated in the prior art. Nor does it necessarily mean that, in the relevant field, it is widely known, well-understood, or routinely used.

While the present disclosure has been described herein with respect to certain illustrated embodiments, those of ordinary skill in the art will recognize and appreciate that the present invention is not so limited. Rather, many additions, deletions, and modifications to the illustrated and described embodiments may be made without departing from the scope of the invention as hereinafter claimed along with their legal equivalents. In addition, features from one embodiment may be combined with features of another embodiment while still being encompassed within the scope of the invention as contemplated by the inventor.

1.-17. (canceled)

18. A method of setting up a trusted communication between devices to form at least part of a medication therapy system, the method comprising:
generating a candidate shared secret key responsive to one or more shared secret parameters; providing the candidate shared secret key to a first therapy device over a first communication link; and
receiving first therapy related information from the first therapy device over a first trusted communication link established responsive to acceptance of a candidate shared secret.

19. The method of claim 18, wherein the one or more shared secret parameters comprise a hash function and a hash key, and generating the candidate shared secret key comprises, at least in part, hashing the hash key using the hash function.

20. The method of claim 19, further comprising generating the hash key responsive to a variable component and a unique identifier component.

21. The method of claim 19, wherein the hash key is a unique identifier associated with the first therapy device.

22. The method of claim 21, wherein the unique identifier is a serial number associated with the first therapy device or a public identifier associated with the first therapy device.

23. The method of claim 18, further comprising: setting up the first communication link as an intermediate communication link; and ending the intermediate communication link responsive to the first trusted communication link being established.

24. The method of claim 18, further comprising receiving over a communication network the one or more shared secret parameters from a remote therapy management system.

25. The method of claim 18, wherein the first therapy device is an analyte monitoring device selected from a group consisting essentially of: a blood glucose meter and a continuous glucose monitor, and the therapy related information comprises blood glucose levels and timing information related to the blood glucose levels.

26. (canceled)

27. The method of claim 18, further comprising sending second therapy related information to a second therapy device, wherein the second therapy information comprises one or more of at least some of the first therapy related information and insulin dosing action information.

28. A remote therapy management system, comprising:
a data store of therapy system records, wherein the therapy system records describe one or more groups of therapy devices;
a data store of therapy system policy definitions that describe restrictions for therapy devices that comprise the groups of therapy devices; and
a policy enforcer configured to receive a request for shared secret parameters from a restricted therapy device and provide the shared secret parameters to the restricted therapy device responsive to the request and the one or more therapy system policies.

29. The remote therapy management system of claim 28, wherein the one or more therapy system policies comprise at least a connection policy that limits a number of therapy devices of a first type of therapy devices with which the restricted therapy device may establish trusted communication links.

30. The remote therapy management system of claim 29, wherein the first type of therapy device is a glucose monitor, and the number of therapy devices of the first type with which the restricted therapy device may establish trusted communication links is one (1).

31. The remote therapy management system of claim 30 wherein the restricted therapy device is configured to receive therapy related information from the glucose monitor.

32. The remote therapy management system of claim 29, wherein the first type of therapy device is an insulin delivery device, and the number of therapy devices of the first type with which the restricted therapy device may establish trusted communication links is one.

33. (canceled)

34. The remote therapy management system of claim 28, wherein the policy enforcer is configured to:
identify a first group of therapy devices of the one or more groups of therapy devices responsive to an identity of the restricted therapy device; and
retrieve the shared secret parameters responsive to the identified first group of therapy devices.

35. The remote therapy management system of claim 34, wherein the policy enforcer is configured to reject the request for the shared secret parameters responsive to the one or more therapy system policies.

36. The remote therapy management system of claim 34, wherein the policy enforcer is configured to reject the request responsive to a determination that the restricted therapy device is not part of a group of therapy devices.

37. The remote therapy management system of claim 34, wherein the policy enforcer is configured to reject the request responsive to a determination that the restricted therapy device is part of a group of therapy devices, and that the group of therapy devices does not include any therapy devices with which the restricted therapy device is permitted to establish a trusted communication link.

38. A method of enforcing a connection policy for a therapy system, comprising:
receiving a request for shared secret parameters from a first therapy device trying to establish a trusted communication link with a second therapy device; and
replying to the request for shared secret parameters responsive to one or more of therapy system policies.

39. The method of claim 38, further comprising:
identifying a policy that would be violated by establishing the trusted communication link; and
sending a reply message that indicates the request for shared secret parameters has been rejected.
